(19) United States
(12) Patent Application Publication
(10) Pub. No.: US 2009/0036509 A1
(43) Pub. Date: Feb. 5, 2009

(54) N-(Ortho-Phenyl)-1-Methyl -3-Trifluoromethlpyrazole-4-Carboxanilides and Their Use as Fungicides

(75) Inventors: Markus Gewehr, Kastellaun (DE); Bernd Muller, Frankenthal (DE); Thomas Grote, Wachenheim (DE); Wassilios Grammenos, Ludwigshafen (DE); Anja Schwogler, Mannheim (DE); Joachim Rheinheimer, Ludwigshafen (DE); Carsten Blettner, Hong Kong (CN); Peter Schafer, Ottersheim (DE); Frank Schieweck, Hessheim (DE); Oliver Wagner, Neustadt (DE); Frank Werner, Neustadt (DE); Jan Rether, Kaiserslautern (DE); Siegfried Strathmann, Limburgerhof (DE); Reinhard Stierl, Freinsheim (DE); Maria Scherer, Godramstein (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 11/628,324

(22) PCT Filed: Jun. 11, 2005
(86) PCT No.: PCT/EP2005/006278

§ 371 Date: Aug. 28, 2008

(30) Foreign Application Priority Data
Jun. 18, 2004 (DE) 10 2004 029 468.2

(51) Int. Cl. A01N 43/56; C07D 231/14; A01P 3/00

(52) U.S. Cl. 514/406; 548/374.1

(57) ABSTRACT

The present invention relates to N-(ortho-phenyl)-1-methyl-3-trifluoromethylpyrazole-4-carboxanilides of the formula I

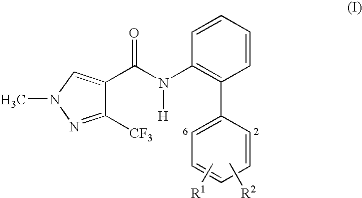

in which the substituents are as defined below:
R¹ and R² independently of one another are halogen, C₁-C₆-alkyl, C₁-C₆-haloalkyl, cyano, nitro, methoxy, trifluoromethoxy or difluoromethoxy;
with the proviso that when R² is chlorine in position 4, R¹ is not trifluoromethyl in position 3.

The present invention relates to N-(ortho-phenyl)-1-methyl-3-trifluoromethylpyrazole-4-carboxanilides of the formula I

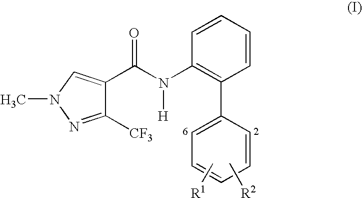

in which the substituents are as defined below:
R¹ and R² independently of one another are halogen, C₁-C₆-alkyl, C₁-C₆-haloalkyl, cyano, nitro, methoxy, trifluoromethoxy or difluoromethoxy;
   with the proviso that when R² is chlorine in position 4, R¹ is not trifluoromethyl in position 3.

Moreover, the invention relates to a process for controlling harmful fungi using the compounds I and to the use of the compounds I for preparing fungicidal compositions.

N-(ortho-phenyl)-1-methyl-3-trifluoromethylpyrazole-4-carboxanilides are known from EP-A 0589301 which also discloses a process for their preparation and a list of possible mixing partners from the group of the fungicides, bactericides, acaricides, nematicides and insecticides.

WO 01/42223 likewise discloses N-(ortho-phenyl)-1-methyl-3-trifluoromethylpyrazole-4-carboxanilides which are monosubstituted at the phenyl ring.

JP 09 132567 discloses N-(ortho-phenyl)-1-methyl-3-trifluoromethylpyrazole-4-carboxanilides which are substituted at the phenyl ring by trifluoromethyl.

However, the 1-methyl-3-trifluoromethylpyrazole-4-carboxanilides described are, in particular at low application rates, not entirely satisfactory.

It was an object of the present invention to provide novel 1-methyl-3-trifluoromethyl-pyrazole-4-carboxanilides having improved fungicidal action, in particular at low application rates.

Accordingly, we have found the compounds of the formula I defined at the outset.

Preference is given to N-(ortho-phenyl)-1-methyl-3-trifluoromethylpyrazole-4-carboxanilides of the formula I

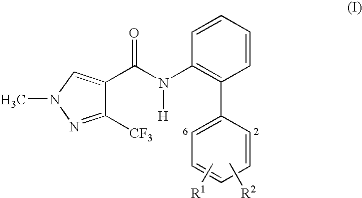

in which the substituents are as defined below:

R¹ and R² independently of one another are fluorine, chlorine, cyano, methyl, methoxy or trifluoromethyl.

Preference is furthermore given to compounds of the formula I in which R¹ and R² independently of one another are fluorine, chlorine, cyano or methoxy.

Particular preference is given to compounds of the formula I in which R¹ and R² independently of one another are fluorine or chlorine.

Very particular preference is given to compounds of the formula I in which R¹ and R² are located in the 3- and 4-positions of the phenyl ring.

From among the compounds I according to the invention, preference is given to compounds of the formulae Ia to If listed in the tables below.

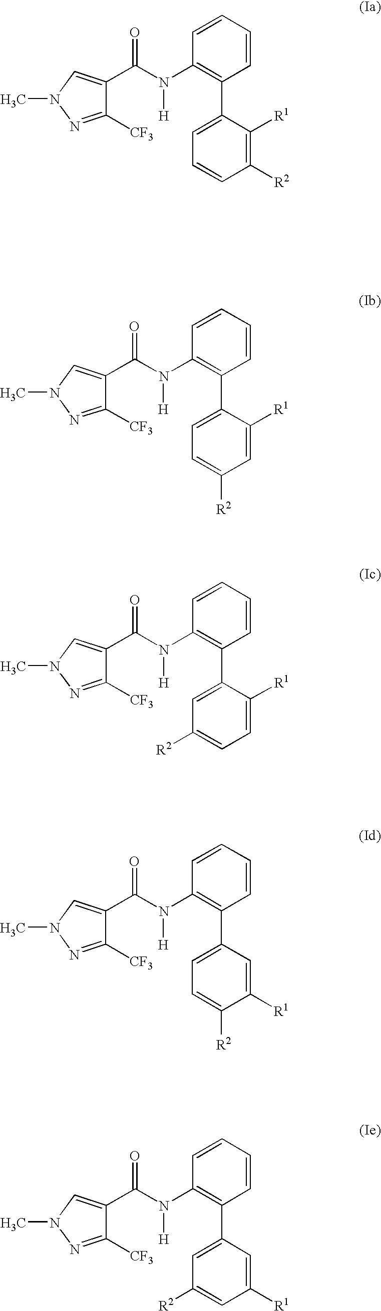

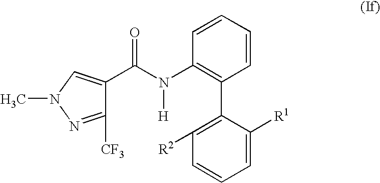

TABLE A

| Number | R² |
|---|---|
| 1 | fluorine |
| 2 | chlorine |
| 3 | bromine |
| 4 | iodine |
| 5 | methyl |
| 6 | methoxy |
| 7 | trifluoromethyl |
| 8 | trifluoromethoxy |
| 9 | cyano |
| 10 | nitro |
| 11 | difluoromethoxy |

Table 1:
Compound 1.1-1.11
Compounds of the formula Ia in which R¹ is fluorine and R² has in each case one of the
meanings of Table A.
Table 2:
Compound 2.1-2.11
Compounds of the formula Ia in which R¹ is chlorine and R² has in each case one of
the meanings of Table A.
Table 3:
Compound 3.1-3.11
Compounds of the formula Ia in which R¹ is bromine and R² has in each case one of
the meanings of Table A.
Table 4:
Compound 4.1-4.11
Compounds of the formula Ia in which R¹ is iodine and R² has in each case one of the
meanings of Table A.
Table 5:
Compound 5.1-5.11
Compounds of the formula Ia in which R¹ is methyl and R² has in each case one of the
meanings of Table A.
Table 6:
Compound 6.1-6.11
Compounds of the formula Ia in which R¹ is methoxy and R² has in each case one of
the meanings of Table A.
Table 7:
Compound 7.1-7.11
Compounds of the formula Ia in which R¹ is trifluoromethyl and R² has in each case
one of the meanings of Table A.
Table 8:
Compound 8.1-8.11
Compounds of the formula Ia in which R¹ is trifluoromethoxy and R² has in each case
one of the meanings of Table A.
Table 9:
Compound 9.1-9.11
Compounds of the formula Ia in which R¹ is cyano and R² has in each case one of the
meanings of Table A.
Table 10:
Compound 10.1-10.11
Compounds of the formula Ia in which R¹ is nitro and R² has in each case one of the
meanings of Table A.
Table 11:
Compound 11.1-11.11
Compounds of the formula Ib in which R¹ is difluoromethoxy and R² has in each case
one of the meanings of Table A.
Table 12
Compound 12.1-12:.11
Compounds of the formula Ib in which R¹ is fluorine and R² has in each case one of the
meanings of Table A.
Table 13:
Compound 13.1-13.11
Compounds of the formula Ib in which R¹ is chlorine and R² has in each case one of
the meanings of Table A.
Table 14:
Compound 14.1-14.11
Compounds of the formula Ib in which R¹ is bromine and R² has in each case one of
the meanings of Table A.
Table 15:
Compound 15.1-15.11
Compounds of the formula Ib in which R¹ is iodine and R² has in each case one of the
meanings of Table A.
Table 16:
Compound 16.1-16.11
Compounds of the formula Ib in which R¹ is methyl and R² has in each case one of the
meanings of Table A.
Table 17:
Compound 17.1-17.11
Compounds of the formula Ib in which R¹ is methoxy and R² has in each case one of
the meanings of Table A.
Table 18:
Compound 18.1-18.11
Compounds of the formula Ib in which R¹ is trifluoromethyl and R² has in each case
one of the meanings of Table A.
Table 19:
Compound 19.1-19.11
Compounds of the formula Ib in which R¹ is trifluoromethoxy and R² has in each case
one of the meanings of Table A.
Table 20:
Compound 20.1-20.11
Compounds of the formula Ib in which R¹ is cyano and R² has in each case one of the
meanings of Table A.
Table 21:
Compound 21.1-21.11
Compounds of the formula Ib in which R¹ is nitro and R² has in each case one of the
meanings of Table A.
Table 22:
Compound 22.1-22.11
Compounds of the formula Ib in which R¹ is difluoromethoxy and R² has in each case
one of the meanings of Table A.
Table 23:
Compound 23.1-23.11
Compounds of the formula Ic in which R¹ is fluorine and R² has in each case one of the
meanings of Table A.
Table 24:
Compound 24.1-24.11
Compounds of the formula Ic in which R¹ is chlorine und R² has in each case one of
the meanings of Table A.
Table 25:
Compound 25.1-25.11
Compounds of the formula Ic in which R¹ is bromine and R² has in each case one of
the meanings of Table A.
Table 26:
Compound 26.1-26.11
Compounds of the formula Ic in which R¹ is iodine and R² has in each case one of the
meanings of Table A.
Table 27:
Compound 27.1-27.11
Compounds of the formula Ic in which R¹ is methyl and R² has in each case one of the
meanings of Table A.
Table 28:
Compound 28.1-28.11
Compounds of the formula Ic in which R¹ is methoxy and R² has in each case one of
the meanings of Table A.
Table 29:
Compound 29.1-29.11
Compounds of the formula Ic in which R¹ is trifluoromethyl and R² has in each case one
of the meanings of Table A.
Table 30:
Compound 30.1-30.11
Compounds of the formula Ic in which R¹ is trifluoromethoxy and R² has in each case
one of the meanings of Table A.
Table 31:
Compound 31.1-31.11
Compounds of the formula Ic in which R¹ is cyano and R² has in each case one of the
meanings of Table A.
Table 32:
Compound 32.1-32.11
Compounds of the formula Ic in which R¹ is nitro and R² has in each case one of the
meanings of Table A.
Table 33:
Compound 33.1-33.11
Compounds of the formula Ic in which R¹ is difluoromethoxy and R² has in each case
one of the meanings of Table A.
Table 34:
Compound 34.1-34.11
Compounds of the formula Id in which R¹ is fluorine and R² has in each case one of the
meanings of Table A.
Table 35:
Compound 35.1-35.11
Compounds of the formula Id in which R¹ is chlorine and R² has in each case one of
the meanings of Table A.
Table 36:
Compound 36.1-36.11
Compounds of the formula Id in which R¹ is bromine and R² has in each case one of
the meanings of Table A.
Table 37:
Compound 37.1-37.11
Compounds of the formula Id in which R¹ is iodine and R² has in each case one of the
meanings of Table A.
Table 38:
Compound 38.1-38.11
Compounds of the formula Id in which R¹ is methyl and R² has in each case one of the
meanings of Table A.
Table 39:
Compound 39.1-39.11
Compounds of the formula Id in which R¹ is methoxy and R² has in each case one of
the meanings of Table A.
Table 40:
Compound 40.1-40.10
Compounds of the formula Id in which R¹ is trifluoromethyl and R² has in each case
one of the meanings of Table A, except for the meaning having the number 2.
Table 41:
Compound 41.1-41.11
Compounds of the formula Id in which R¹ is trifluoromethoxy and R² has in each case
one of the meanings of Table A.
Table 42:
Compound 42.1-42.11
Compounds of the formula Id in which R¹ is cyano and R² has in each case one of the
meanings of Table A.
Table 43:
Compound 43.1-43.11
Compounds of the formula Id in which R¹ is nitro and R² has in each case one of the
meanings of Table A.
Table 44:
Compound 44.1-44.11
Compounds of the formula Id in which R¹ is difluoromethoxy and R² has in each case
one of the meanings of Table A.
Table 45:
Compound 45.1-45.11
Compounds of the formula Ie in which R¹ is fluorine and R² has in each case one of the
meanings of Table A.
Table 46:
Compound 46.1-46.11
Compounds of the formula Ie in which R¹ is chlorine and R² has in each case one of
the meanings of Table A.
Table 47:
Compound 47.1-47.11
Compounds of the formula Ie in which R¹ is bromine and R² has in each case one of
the meanings of Table A.
Table 48:
Compound 48.1-48.11
Compounds of the formula Ie in which R¹ is iodine and R² has in each case one of the
meanings of Table A.
Table 49:
Compound 49.1-49.11
Compounds of the formula Ie in which R¹ is methyl and R² has in each case one of the
meanings of Table A.
Table 50:
Compound 50.1-50.11
Compounds of the formula Ie in which R¹ is methoxy and R² has in each case one of
the meanings of Table A.
Table 51:
Compound 51.1-51.11
Compounds of the formula Ie in which R¹ is trifluoromethyl and R² has in each case
one of the meanings of Table A.
Table 52:
Compound 52.1-52.11
Compounds of the formula Ie in which R¹ is trifluoromethoxy and R² has in each case
one of the meanings of Table A.
Table 53:
Compound 53.1-53.11
Compounds of the formula Ie in which R¹ is cyano and R² has in each case one of the
meanings of Table A.
Table 54:
Compound 54.1-54.11
Compounds of the formula Ie in which R¹ is nitro and R² has in each case one of the
meanings of Table A.
Table 55:
Compound 55.1-55.11
Compounds of the formula Ie in which R¹ is difluoromethoxy and R² has in each case
one of the meanings of Table A.
Table 56:
Compound 56.1-56.11
Compounds of the formula If in which R¹ is fluorine and R² has in each case one of the
meanings of Table A.
Table 57:
Compound 57.1-57.11
Compounds of the formula If in which R¹ is chlorine and R² has in each case one of the
meanings of Table A.
Table 58:
Compound 58.1-58.11
Compounds of the formula If in which R¹ is bromine and R² has in each case one of the
meanings of Table A.
Table 59:
Compound 59.1-59.11
Compounds of the formula If in which R¹ is iodine and R² has in each case one of the
meanings of Table A.
Table 60:
Compound 60.1-60.11
Compounds of the formula If in which R¹ is methyl and R² has in each case one of the
meanings of Table A.
Table 61:
Compound 61.1-61.11
Compounds of the formula If in which R¹ is methoxy and R² has in each case one of
the meanings of Table A.
Table 62:
Compound 62.1-62.11
Compounds of the formula If in which R¹ is trifluoromethyl and R² has in each case one
of the meanings of Table A.
Table 63:
Compound 63.1-63.11
Compounds of the formula If in which R¹ is trifluoromethoxy and R² has in each case
one of the meanings of Table A.
Table 64:
Compound 64.1-64.11
Compounds of the formula If in which R¹ is cyano and R² has in each case one of the
meanings of Table A.
Table 65:
Compound 65.1-65.11
Compounds of the formula If in which R¹ is nitro and R² has in each case one of the
meanings of Table A.
Table 66:
Compound 66.1-66.11
Compounds of the formula If in which R¹ is difluoromethoxy and R² has in each case
one of the meanings of Table A.

In the context of the present invention, halogen denotes fluorine, chlorine, bromine and iodine and in particular fluorine and chlorine.

The term “alkyl” includes straight-chain and branched alkyl groups. They are preferably straight-chain and branched C₁-C₆-alkyl groups. Examples of alkyl groups are alkyl such as, in particular, methyl, ethyl, propyl, 1-methylethyl, butyl, 1-methylpropyl, 2-methylpropyl 1,1-dimethylethyl.

Haloalkyl is an alkyl group as defined above which is partially or fully halogenated by one or more halogen atoms, in particular fluorine and chlorine. Preferably, 1 to 3 halogen atoms are present, and the difluoromethyl and the trifluoromethyl groups are particularly preferred.

Processes for preparing the compounds of the formula F are known from EP-A 0 589 301.

For example, 1-methyl-3-trifluoromethylpyrazolecarbonyl halides of the formula II are reacted with an aniline of the formula III to give the compounds of the formula I:

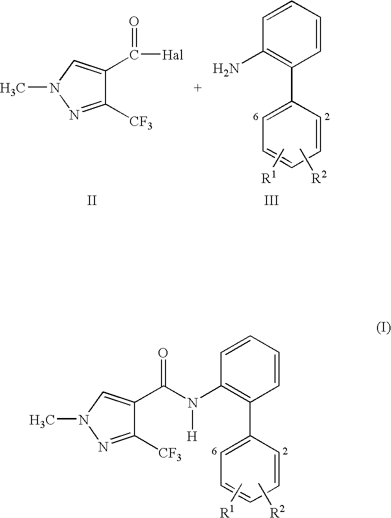

The radical Hal in the formula II denotes a halogen atom, such as chlorine, bromine and iodine, in particular chlorine or bromine.

This reaction is usually carried out at temperatures of from −20° C. to 100° C., preferably from 0° C. to 50° C.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol and also dimethyl sulfoxide and dimethylformamide, particularly preferably toluene and tetrahydrofuran.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide und calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate and calcium carbonate, and also alkali metal bicarbonates, such as sodium bicarbonate, and organometallic compounds, in particular alkali metal alkyls, such as methyllithium, butyllithium and phenyllithium, alkylmagnesium halides, such as methylmagnesium chloride, and also alkali metal und alkaline earth metal alkoxides, such as sodium methoxide, sodium ethoxide, potassium ethoxide, potassium tert.-butoxide and dimethoxymagnesium, moreover organic bases, for example tertiary amines, such as trimethylamine, triethylamine, triisopropylamine and N-methylpiperidine, pyridine, substituted pyridines, such as collidine, lutidine and 4-dimethylaminopyridine, and also bicyclic amines.

Particular preference is given to using triethylamine and pyridine.

The bases are generally employed in equimolar amounts, based on the compound II. However, they can also be employed in an excess of from 5 mol % to 30 mol %, preferably 5 mol % to 10 mol %, or—in the case of tertiary amines—as solvents, if appropriate.

The starting materials are generally reacted with one another in equimolar amounts. In terms of yield, it may be advantageous to employ II in an excess of from 1 mol % to 20 mol %, preferably 1 mol % to 10 mol %, based on III.

The compounds I according to the invention can, in the application form as fungicides, also be present together with other active compounds, e.g. with herbicides, insecticides, growth regulators, fungicides or also with fertilizers. On mixing the compounds I or the compositions comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but does not limit them:
sulfur, dithiocarbamates and their derivatives, such as iron(III) dimethyldithio-carbamate, zinc dimethyldithiocarbamate, zinc ethylenebisdithiocarbamate, manganese ethylenebisdithiocarbamate, manganese zinc ethylenediaminebis-dithiocarbamate, tetramethylthiuram disulfide, ammonia complex of zinc (N,N′-ethylenebisdithiocarbamate), ammonia complex of zinc (N,N′-propylene-bisdithiocarbamate), zinc (N,N′-propylenebisdithiocarbamate) or N,N′-poly-propylenebis(thiocarbamoyl)disulfide;
nitro derivatives, such as dinitro(1-methylheptyl)phenyl crotonate, 2-sec-butyl-4,6-dinitrophenyl 3,3-dimethylacrylate, 2-sec-butyl-4,6-dinitrophenyl isopropyl carbonate or diisopropyl 5-nitroisophthalate;
heterocyclic substances, such as 2-heptadecyl-2-imidazoline acetate, 2,4-dichloro-6-(o-chloroanilino)-s-triazine, O,O-diethyl phthalimidophosphono-thioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-(methoxycarbonylamino)benz-imidazole, 2-(2-furyl)benzimidazole, 2-(4-thiazolyl)benzimidazole, N-(1,1,2,2-tetrachloroethylthio)tetrahydrophthalimide, N-(trichloromethylthio)tetra-hydrophthalimide or N-(trichloromethylthio)phthalimide,
N-dichlorofluoromethylthio-N′,N′-dimethyl-N-phenylsulfuric acid diamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, 2-thiopyridine 1-oxide, 8-hydroxyquinoline or its copper salt, 2,3-dihydro-5-carboxanilido-6-methyl-1,4-oxathiin, 2,3-dihydro-5-carboxanilido-6-methyl-1,4-oxathiin 4,4-dioxide, 2-methyl-5,6-dihydro-4H-pyran-3-carboxamide, 2-methylfuran-3-carboxanilide, 2,5-dimethylfuran-3 carboxanilide, 2,4,5-trimethyl-furan-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine 2,2,2-trichloroethyl acetal, piperazine-1,4-diylbis-1-(2,2,2-trichloroethyl)formamide, 1-(3,4-dichloroanilino)-1-formyl-amino-2,2,2-trichloroethane, 2,6-dimethyl-N-tridecylmorpholine or its salts, 2,6-dimethyl-N-cyclododecylmorpholine or its salts, N-[3-(p-(tert-butyl)phenyl)-2-methylpropyl]-cis-2,6-dimethylmorpholine, N-[3-(p-(tert-butyl)phenyl)-2-methyl-propyl]piperidine, 1-[2-(2,4-dichlorophenyl)-4-ethyl-1,3-dioxolan-2-ylethyl]-1H-1,2,4-triazole, 1-[2-(2,4-dichlorophenyl)-4-(n-propyl)-1,3-dioxolan-2-ylethyl]-1H-1,2,4-triazole, N-(n-propyl)N-(2,4,6-trichlorophenoxyethyl)-N′-imidazolylurea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)oxiran-2-ylmethyl]-1H-1,2,4-triazole, α-(2-chlorophenyl)-α-(4-chlorophenyl)5-pyrimidine methanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene or 1,2-bis(3-methoxycarbonyl-2-thioureido)benzene,
strobilurins, such as methyl E-methoxyimino[α-(o-tolyloxy)-o-tolyl]acetate, methyl E-2-{2-[6-(2-cyanophenoxy)pyrimidin-4-yloxy]phenyl}3-methoxyacrylate, methyl E-methoxyimino-[α-(2-phenoxyphenyl)]acetamide, methyl E-methoxyimino-[α-(2,5-dimethylphenoxy)-o-tolyl]acetamide,
anilinopyrimidines, such as N-(4,6-dimethylpyrimidin-2-yl)aniline, N-[4-methyl-6-(1-propynyl)pyrimidin-2-yl]aniline or N-[4-methyl-6-cyclopropylpyrimidin-2-yl]-aniline,
phenylpyrroles, such as 4-(2,2-difluoro-1,3-benzodioxol-4-yl)pyrrole-3-carbonitrile,
cinnamamides, such as 3-(4-chlorophenyl)-3-(3,4-dimethoxyphenyl)acryloyl-morpholine,
and various fungicides, such as dodecylguanidine acetate, 3-[3-(3,5-dimethyl-2-oxycyclohexyl) 2-hydroxyethyl]glutarimide, hexachlorobenzene, methyl N-(2,6-dimethylphenyl)-N-(2-furoyl)-DL-alaninate, N-(2,6-dimethylphenyl)-N-(2′-methoxy-acetyl)-DL-alanine methyl ester, N-(2,6-dimethylphenyl)-N-chloroacetyl-D,L-2-aminobutyrolactone, N-(2,6-dimethylphenyl)-N-(phenylacetyl)-DL-alanine methyl ester, 5-methyl-5-vinyl-3-(3,5-dichlorophenyl) 2,4-dioxo-1,3-oxazolidine, 3-(3,5-dichlorophenyl)-5-methyl-5-methoxymethyl-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-N-(ethylaminocarbonyl)-2-[methoxyimino]acetamide, 1-[2-(2,4-dichlorophenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

The compounds of the formula I are distinguished by being highly active against a wide range of phytopathogenic fungi, in particular from the classes of the Ascomycetes, Basidiomycetes, Phycomycetes and Deuteromycetes. Some of them act systemically and can therefore also be used as foliar and soil-acting fungicides. They can also be employed for seed-dressing.

They are particularly important in the control of a multitude of fungi on various cultivated plants, such as cotton, vegetable species (for example cucumbers, beans, tomatoes, potatoes and cucurbits), barley, grass, oats, bananas, coffee, corn, fruit species, rice, rye, soya, grapevines, wheat, ornamental plants, sugar cane and also on a large number of seeds.

They are particularly suitable for controlling the following phytopathogenic fungi: Blumeria graminis (powdery mildew) on cereals, Erysiphe cichoracearum and Sphaerotheca fuliginea on cucurbits, Podosphaera leucotricha on apples, Uncinula necator on grapevines, Puccinia species on cereals, Rhizoctonia species on cotton, rice and lawns, Ustilago species on cereals and sugar cane, Venturia inaequalis (scab) on apples, Helminthosporium species on cereals, Septoria nodorum on wheat, Botrytis cinerea (gray mold) on strawberries, vegetables, ornamental plants and grapevines, Cercospora arachidicola on peanuts, Pseudocercosporefla herpotrichoides on wheat and barley, Pyricularia oryzae on rice, Phytophthora infestans on potatoes and tomatoes, Plasmopara viticola on grapevines, Pseudoperonospora species on hops and cucumbers, Alternaria species on fruit and vegetables, Mycosphaerelia species on bananas and also Fusarium and Verticillium species.

Depending on the desired effect, the application rates of the compounds of the formula I according to the invention are, in particular in the case of areas under agricultural cultivation, from 0.01 to 8 kg/ha, preferably from 0.1 to 5 kg/ha, in particular from 0.1 to 3.0 kg/ha.

For the compound I, the application rates are in particular from 0.01 to 1 kg/ha, preferably from 0.05 to 0.5 kg/ha, in particular from 0.05 to 0.3 kg/ha.

In seed-dressing, application rates of mixture are generally from 0.001 to 250 g/kg of seed, preferably from 0.01 to 100 g/kg, in particular from 0.01 to 50 g/kg.

In the control of phytopathogenic harmful fungi, the application of the compound I is carried out by spraying or dusting the seeds, the plants or the soils before or after sowing of the plants or before or after emergence of the plants.

The fungicidal compounds I according to the invention can be prepared, for example, in the form of directly sprayable solutions, powders and suspensions or in the form of highly concentrated aqueous, oily or other suspensions, dispersions, emulsions, oil dispersions, pastes, dustable products, compositions for broadcasting or granules and be applied by spraying, atomising, dusting, broadcasting or pouring. The use form depends on the particular intended purpose; in each case, it should ensure as fine and even a distribution as possible of the mixture according to the invention.

The formulations are prepared in a manner known per se, for example by adding solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers, such as ground natural minerals (for example kaolins, clays, talc, chalk) and ground synthetic minerals (for example highly disperse silica, silicates); emulsifiers, such as nonionic and anionic emulsifiers (for example polyoxyethylene fatty alcohol ethers, alkylsulfonates and arylsulfonates), and dispersants, such as lignosulfite waste liquors and methylcellulose.

Suitable surfactants are alkali metal, alkaline earth metal and ammonium salts of aromatic sulfonic acids, for example lignosulfonic acid, phenolsulfonic acid, naphthalenesulfonic acid, and dibutylnaphthalenesulfonic acid, and of fatty acids, alkylsulfonates and alkylarylsulfonates, alkyl sulfates, laurylether sulfates, fatty alcohol sulfates, and salts of sulfated hexa-, hepta- and octadecanols or fatty alcohol glycol ethers, condensates of sulfonated naphthalene and naphthalene derivatives with formaldehyde, condensates of naphthalene or of naphthalenesulfonic acids with phenol and formaldehyde, polyoxyethylene octylphenyl ethers, ethoxylated isooctylphenol, octylphenol or nonylphenol, alkylphenyl polyglycol ethers or tributylphenyl polyglycol ethers, alkylaryl polyether alcohols, isotridecyl alcohol, fatty alcohol ethylene oxide condensates, ethoxylated castor oil, polyoxyethylene alkyl ethers or polyoxypropylene, lauryl alcohol polyglycol ether acetate, sorbitol esters, lignosulfite waste liquors or methylcellulose.

Powders, compositions for broadcasting and dustable products can be prepared by mixing or concomitantly grinding the compound I with a solid carrier.

Granules (for example coated granules, impregnated granules and homogeneous granules) are usually prepared by binding the active compound(s) onto a solid carrier.

Examples of fillers and solid carriers are mineral earths, such as silica gels, silica, silicates, talc, kaolin, limestone, lime, chalk, bole, loess, clay, dolomite, diatomaceous earth, calcium sulfate, magnesium sulfate, magnesium oxide, ground synthetic materials, and fertilizers, such as ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.1 to 95% by weight, preferably from 0.5 to 90% by weight, of the compound I.

The active compounds are employed in a purity of from 90% to 100%, preferably 95% to 100% (according to the NMR spectrum or HPLC).

The following are examples of formulations: 1. Products for dilution with water

### A) Water-Soluble Concentrates (SL)

10 parts by weight of the active compounds are dissolved in water or in a water-soluble solvent. As an alternative, wetters or other auxiliaries are added. The active compound dissolves upon dilution with water.

### B) Dispersible Concentrates (DC)

20 parts by weight of the active compounds are dissolved in cyclohexanone with addition of a dispersant, for example polyvinylpyrrolidone. Dilution with water gives a dispersion.

### C) Emulsifiable Concentrates (EC)

15 parts by weight of the active compounds are dissolved in xylene with addition of calcium dodecylbenzenesulfonate and castor oil ethoxylate (in each case 5% strength). Dilution with water gives an emulsion.

### D) Emulsions (EW, EO)

40 parts by weight of the active compounds are dissolved in xylene with addition of calcium dodecylbenzenesulfonate and castor oil ethoxylate (in each case 5% strength). This mixture is introduced into water by means of an emulsifying machine (Ultraturax) and made into a homogeneous emulsion. Dilution with water gives an emulsion.

### E) Suspensions (SC, OD)

In an agitated ball mill, 20 parts by weight of the active compounds are comminuted with addition of dispersants and wetters and water or an organic solvent to give a fine active compound suspension. Dilution with water gives a stable suspension of the active compound.

### F) Water-Dispersible Granules and Water-Soluble Granules (WG, SG)

50 parts by weight of the active compounds are ground finely with addition of dispersants and wetters and prepared as water-dispersible or water-soluble granules by means of technical appliances (for example extrusion, spray tower, fluidized bed). Dilution with water gives a stable dispersion or solution of the active compound.

### G) Water-Dispersible Powders and Water-Soluble Powders (WP, SP)

75 parts by weight of the active compounds are ground in a rotor-stator mill with addition of dispersants and wetters and silica gel. Dilution with water gives a stable dispersion or solution of the active compound.

### 2. Products to be Applied Undiluted

### H) Dustable Powders (DP)

5 parts by weight of the active compounds are ground finely and mixed intimately with 95% of finely divided kaolin. This gives a dustable product.

### I) Granules (GR, FG, GG, MG)

0.5 part by weight of the active compounds is ground finely and combined with 95.5% of carriers. Current methods are extrusion, spray-drying or the fluidized bed. This gives granules to be applied undiluted.

### J) ULV Solutions (UL)

10 parts by weight of the active compounds are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, compositions for broadcasting, or granules, by means of spraying, atomizing, dusting, broadcasting or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil which are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even to apply the active compound without additives.

Oils of various types, wetters, adjuvants, herbicides, fungicides, other pesticides, bactericides may be added to the active compounds, even, if appropriate, not until immediately prior to use (tank mix). These agents can be admixed with the compositions according to the invention in a weight ratio of from 1:10 to 10:1.

The compound I or the corresponding formulations are applied by treating the harmful fungi, their habitat or the plants, seeds, soils, areas, materials or spaces to be kept free from them with a fungicidally effective amount of the compound I.

Application can be before or after infection by the harmful fungi.

### PREPARATION EXAMPLES

### Example 1

### 1.) Synthesis of ortho-(3,4-dichlorophenyl)aniline

0.14 g of tetrakistriphenylphosphinepalladium(0) was added to a solution of 20.47 g of 2-bromoaniline, 24.98 g of 3,4-dichlorophenylboronic acid and 25.23 g of sodium carbonate in a mixture of 150 ml of water and 450 ml of ethylene glycol dimethyl ether. The mixture was stirred under reflux for 48 hours. The mixture was concentrated under reduced pressure. The residue was taken up in methyl tert-butyl ether, washed once with sodium bicarbonate solution and four times with water, dried over sodium sulfate and concentrated under reduced pressure. Chromatographic purification using a mixture of toluene and cyclohexane (1:2) gave 15.5 g of the product as a light-yellow powder.

### 2.) Synthesis of N-(ortho(3,4-dichlorophenyl)phenyl)-1-methyl-3-trifluoromethyl-pyrazole-4-carboxamide

0.32 g of 1-methyl-3-trifluoromethylpyrazole-4-carbonyl chloride, 0.36 g of ortho(3,4-dichlorophenyl)aniline and 0.23 g of triethylamine were dissolved in 10 ml of toluene. The mixture was stirred at room temperature for 4 h, 20 ml of methyl tert-butyl ether were then added and the mixture was washed twice with 5% strength hydrochloric acid, twice with 5% strength aqueous sodium hydroxide solution and once with brine. The organic phase was dried over sodium sulfate and concentrated under reduced pressure. Chromatographic purification using a mixture of toluene and methyl tert-butyl ether gave 0.32 g of the product as a colorless powder. Mp=131-133° C.

TABLE 67

| | |
|---|---|
| | (I) |
| 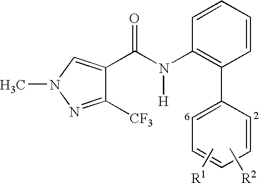 | |

| Comp. | R² | R¹ | |
|---|---|---|---|
| | | | mp |
| 67.1 | 3-Cl | 4-Cl | 131-133° C. |
| 67.2 | 3-Cl | 4-F | 133-134° C. |
| 67.3 | 2-Cl | 4-Cl | 136-140° C. |
| 67.4 | 3-F | 4-F | 126-128° C. |
| 67.5 | 2-Cl | 5-Cl | 101-105° C. |
| 67.6 | 2-F | 5-F | 110-112° C. |
| 67.7 | 2-F | 4-Cl | 136-138° C. |
| 67.8 | 2-CH₃ | 4-Cl | 130-131° C. |
| 67.9 | 3-CH₃ | 4-Cl | 109-111° C. |
| 67.10 | 2-CH₃ | 4-F | 125-126° C. |
| 67.11 | 3-CH₃ | 4-F | 126-127° C. |
| 67.12 | 3-F | 4-Cl | 149-150° C. |
| 67.13 | 2-F | 4-F | 100-102° C. |
| 67.14 | 2-F | 4-OCH₃ | 102-104° C. |
| 67.15 | 2-F | 6-F | 140-143° C. |
| | | | HPLC |
| | | | retention time |
| | | | [min] |
| 67.16 | 3-CH₃ | 5-CH₃ | 3.71 |
| 67.17 | 3-NO₂ | 4-Cl | 3.36 |
| 67.18 | 3-CF₃ | 5-CF₃ | 3.83 |
| 67.19 | 3-CH₃ | 4-CH₃ | 2.99 |
| 67.20 | 3-CF₃ | 4-NO₂ | 3.49 |
| 67.21 | 3-CH₃ | 4-OCH₃ | 3.55 |
| 67.22 | 3-OCH₃ | 4-OCH₃ | 3.03 |
| 67.23 | 3-F | 4-OCH₃ | 3.26 |
| 67.24 | 3-OCH₃ | 4-Cl | 3.46 |

### Use Examples

The active compounds were prepared as a stock solution with 25 mg of active compound which was made up to 10 ml with a mixture of acetone and/or DMSO and the emulsifier Uniperol® EL (wetting agent having emulsifying and dispersing action based on ethoxylated alkyl phenols) in a volume ratio solvent/emulsifier of 99 to 1. The solution was then made up to 100 ml with water. This stock solution was diluted to the active compound concentrations stated below using the solvent/emulsifier/water mixture described.

### Use Example 1

### Activity Against Gray Mold on Bell Pepper Leaves Caused by Botrytis Cinerea Protective Application

Bell pepper seedlings of the cultivar “Neusiedler Ideal Elite” were, after 2-3 leaves were well developed, sprayed to runoff point with an aqueous suspension having the concentration of active compounds stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

| | | Infection in % |
|---|---|---|
| | | at 4 ppm using |
| Compound | Structure | BOTRCI P1 |
| Comp. 37of EP-A05 89 301 | 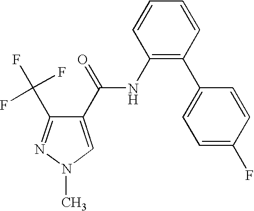 | 70 |
| Comp. 67.4 | 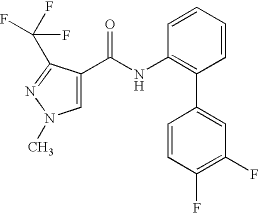 | 40 |
| Untreated | | 90 |

### Use Example 2

### Curative Activity Against Brown Rust of Wheat Caused by Puccinia Recondita

Leaves of potted wheat seedlings of the cultivar “Kanzler” were inoculated with a spore suspension of brown rust (Puccinia recondita). The pots were then placed in a chamber with high atmospheric humidity (90 to 95%) and at 20-22° C. for 24 hours. During this time, the spores germinated and the germ tubes penetrated into the leaf tissue. The next day, the infected plants were sprayed to runoff point with an aqueous suspension having the concentration of active compounds stated below. The suspension or emulsion was prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

| | | Infection in % |
|---|---|---|
| | | at 4 ppm using |
| Compound | Structure | PUCCRT K1 |
| Comp. 37of EP-A05 89 301 | 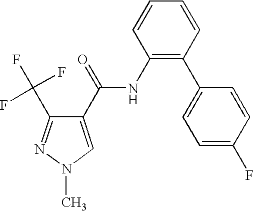 | 30 |
| Comp. 67.4 | 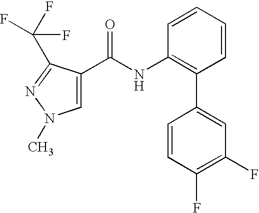 | 5 |
| Untreated | | 90 |

### Use Example 3

### Curative Activity Against Brown Rust of Wheat Caused by Puccinia Recondita

Leaves of potted wheat seedlings of the cultivar “Kanzler” were inoculated with a spore suspension of brown rust (Puccinia recondita). The pots were then placed in a chamber with high atmospheric humidity (90 to 95%) and at 20-22° C. for 24 hours During this time, the spores germinated and the germ tubes penetrated into the leaf tissue. The next day, the infected plants were sprayed to runoff point with an aqueous suspension having the concentration of active compounds stated below. The suspension or emulsion was prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

| | | Infection in % |
|---|---|---|
| | | at 250 ppm |
| | | using |
| Compound | Structure | PUCCRT K1 |
| Comp. 38of EP-A05 89 301 | 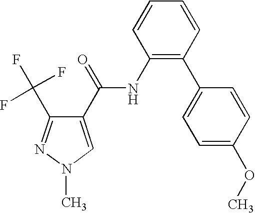 | 60 |
| Comp. 67.14 | 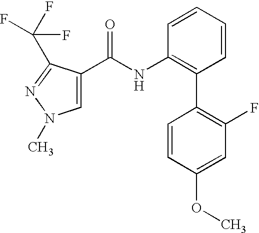 | 7 |
| Untreated | | 90 |

### Use Example 4

### Activity Against Gray Mold on Bell Pepper Leaves Caused by Botrytis Cinerea Protective Application

Bell pepper seedlings of the cultivar “Neusiedler Ideal Elite” were, after 2-3 leaves were well developed, sprayed to runoff point with an aqueous suspension having the concentration of active compounds stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

| | | Infection |
|---|---|---|
| | | in % |
| | | at 4 ppm |
| Com- | | using |
| pound | Structure | BOTRCI P1 |
| Comp. 41of EP-A05 89 301 | 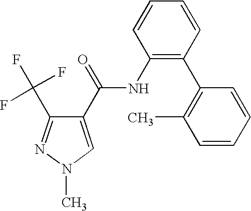 | 25 |
| Comp.67.8 | 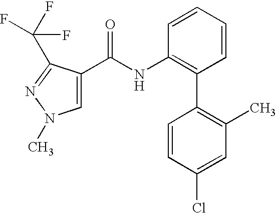 | 0 |
| Untreated | | 90 |

### Use Example 5

### Curative Activity Against Brown Rust of Wheat Caused by Puccinia Recondita

Leaves of potted wheat seedlings of the cultivar “Kanzler” were inoculated with a spore suspension of brown rust (Puccinia recondita). The pots were then placed in a chamber with high atmospheric humidity (90 to 95%) and at 20-22° C. for 24 hours. During this time, the spores germinated and the germ tubes penetrated into the leaf tissue. The next day, the infected plants were sprayed to runoff point with an aqueous suspension having the concentration of active compounds stated below. The suspension or emulsion was prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

| | | Infection in % |
|---|---|---|
| | | at 250 ppm |
| Com- | | using PUCCRT |
| pound | Structure | K1 |
| Comp.41 ofEP-A05 89301 | 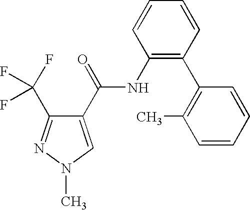 | 60 |
| Comp.67.8 | 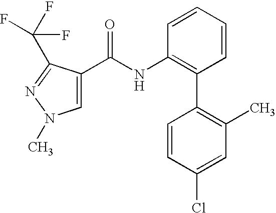 | 0 |
| Un-treated | | 90 |

1. An N-(ortho-phenyl)-1-methyl-3-trifluoromethylpyrazole-4-carboxanilide of the formula I 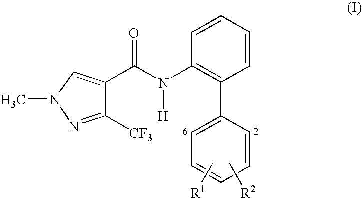
in which the substituents are as defined below:
R¹ is cyano, nitro, halogen, C₁-C₆-alkyl, methoxy, trifluoromethoxy or difluoromethoxy;
R² is cyano, nitro, halogen, C₁-C₆-alkyl, methoxy, trifluoromethoxy or difluoromethoxy.

2. The anilide of the formula I according to claim 1 in which R¹ and R² independently of one another are cyano, fluorine, chlorine, methyl or methoxy.

3. The anilide of the formula I according to claim 1 in which R¹ and R² independently of one another are cyano, fluorine, chlorine or methoxy.

4. The anilide of the formula I according to claim 1 in which R¹ and R² independently of one another are fluorine or chlorine.

5. The anilide of the formula I according to claim 1 in which the substituents R¹ and R² are located in the 3- and 4-positions of the phenyl ring.

6. A method for controlling harmful fungi wherein the harmful fungi, their habitat or the plants, seeds, soils, areas, materials or spaces to be kept free from them are treated with an N-(ortho-phenyl)-1-methyl-3-trifluoromethylpyrazole-4-carboxanilide of the formula I according to claim 1.

7. A fungicidal composition comprising an N-(ortho-phenyl)-1-methyl-3-trifluoromethylpyrazole-4-carboxanilide of the formula I according to claim 1 and a solid or liquid carrier.

8. The use of the compounds I according to claim 1 for controlling phytopathogenic harmful fungi.

9. The anilide of the formula I according to claim 2 in which R¹ and R² independently of one another are cyano, fluorine, chlorine or methoxy.

10. The anilide of the formula I according to claim 2 in which R¹ and R² independently of one another are fluorine or chlorine.

11. The anilide of the formula I according to claim 2 in which the substituents R¹ and R² are located in the 3- and 4-positions of the phenyl ring.

12. The anilide of the formula I according to claim 3 in which the substituents R¹ and R² are located in the 3- and 4-positions of the phenyl ring.

13. The anilide of the formula I according to claim 4 in which the substituents R¹ and R² are located in the 3- and 4-positions of the phenyl ring.

14. A method for controlling harmful fungi wherein the harmful fungi, their habitat or the plants, seeds, soils, areas, materials or spaces to be kept free from them are treated with an N-(ortho-phenyl)-1-methyl-3-trifluoromethylpyrazole-4-carboxanilide of the formula I according to claim 2.

15. A method for controlling harmful fungi wherein the harmful fungi, their habitat or the plants, seeds, soils, areas, materials or spaces to be kept free from them are treated with an N-(ortho-phenyl)-1-methyl-3-trifluoromethylpyrazole-4-carboxanilide of the formula I according to claim 3.

16. A method for controlling harmful fungi wherein the harmful fungi, their habitat or the plants, seeds, soils, areas, materials or spaces to be kept free from them are treated with an N-(ortho-phenyl)-1-methyl-3-trifluoromethylpyrazole-4-carboxanilide of the formula I according to claim 4.

17. A method for controlling harmful fungi wherein the harmful fungi, their habitat or the plants, seeds, soils, areas, materials or spaces to be kept free from them are treated with an N-(ortho-phenyl)-1-methyl-3-trifluoromethylpyrazole-4-carboxanilide of the formula I according to claim 5.

18. A fungicidal composition comprising an N-(ortho-phenyl)-1-methyl-3-trifluoromethylpyrazole-4-carboxanilide of the formula I according to claim 2 and a solid or liquid carrier.

19. A fungicidal composition comprising an N-(ortho-phenyl)-1-methyl-3-trifluoromethylpyrazole-4-carboxanilide of the formula I according to claim 3 and a solid or liquid carrier.

20. A fungicidal composition comprising an N-(ortho-phenyl)-1-methyl-3-trifluoromethylpyrazole-4-carboxanilide of the formula I according to claim 4 and a solid or liquid carrier.
